(19) United States
(12) Patent Application Publication
(10) Pub. No.: US 2026/0272965 A1
(43) Pub. Date: Sep. 17, 2026

(54) SELENOGLYCOSIDE DECOYS OF GLYCOSYLATION FOR ANTI-INFLAMMATORY AND ANTIVIRAL PHARMACEUTICALS

(71) Applicant: Board of Supervisors of Louisiana State University and Agricultural and Mechanical College, Baton Rouge, LA (US)

(72) Inventors: Roger A. Laine, Baton Rouge, LA (US); Justin R. Ragains, Baton Rouge, LA (US); Tiffany Duong, Baton Rouge, LA (US); Brent Stanfield, Baton Rouge, LA (US)

(21) Appl. No.: 19/491,417

(22) PCT Filed: Jun. 5, 2024
(86) PCT No.: PCT/US2024/032529

§ 371 Date: Dec. 9, 2025

Related U.S. Application Data
(60) Provisional application No. 63/507,528, filed on Jun. 12, 2023.

(51) Int. Cl. A61K 31/7034; A61K 31/095; C07H 15/14; C07H 15/18

(52) U.S. Cl. CPC A61K 31/7034; A61K 31/095; C07H 15/14; C07H 15/18

(57) ABSTRACT

In one aspect, the disclosure relates to In accordance with the purpose(s) of the present disclosure, as embodied and broadly described herein, the disclosure, in one aspect, relates to acetylated glycosides comprising at least one O, S, or Se atom external to the glycoside ring, wherein the O, S, or Se is bonded to a moiety selected from C1-C20 linear or branched alkyl, alkenyl, aryl, alkylaryl, or arylalkyl. Also disclosed are methods of making the compounds, pharmaceutical compositions comprising the compounds, and methods of treating or preventing viral diseases and their symptoms in a subject using the same. In one aspect, the glycosides target host cell glycan decoration of viral proteins and do not cause the viruses to mutate. In another aspect, the glycosides and pharmaceutical compositions comprising the same can be applied topically and thereby do not cause systemic side effects in the subject.

### CROSS REFERENCE TO RELATED APPLICATIONS

This application claims the benefit of U.S. Provisional Application No. 63/507,528 filed on Jun. 12, 2023, which is incorporated herein by reference in its entirety.

### BACKGROUND

N- and O-glycosylation are key to trafficking glycoproteins through to proper folding and maturation, through the calnexin calreticulin chaperone system. For example, in the case of antivirals, disrupting N-glycosylation machinery leads to slowing and stopped trafficking of spike proteins including initiating the unfolded protein response, which is crucial for viral infectivity and membrane fusion. Three FDA approved drugs specifically target N-linked glycosylation with low levels of reported systemic side effects: Celgosivir®, Duvoglustat®, and Miglustat®. All three are inhibitors of glycoprotein maturation and have passed FDA safety tests.

There is a crucial need for new virus control approaches. The SARS-CoV-2 pandemic was still causing an average of 350 deaths per day in the United States as of March 2023, after previously killing a million in the US alone. Vaccines help, but are not durable and are beset by frequent viral mutations. Severe COVID still occurs, including pneumonic ARDS, which causes most fatalities, and thus the specter of long COVID looms. The SARS-CoV-2 S19-glycoprotein trimer has 52 N-glycosylation sites that are suitable targets for inhibition of crucial N-glycosylation. Other viruses, including, but not limited to herpes simplex 1 and 2 (HSV-1 and HSV-2, respectively) have coat glycoproteins that may also present N-glycosylation sites.

Despite advances in antiviral therapeutic research, there is still a scarcity of compounds that are potent inhibitors of N- and O-glycosylation of viral proteins and are thus also effective in the treatment of viral diseases including SARS-CoV-2, HSV-1, HSV-2, and others. An ideal antiviral therapeutic compound or composition would have few to no systemic side effects and would be effective against mutated viral variants in addition to the original strains. These needs and other needs are satisfied by the present disclosure. To address the potential for anti-inflammatory effects, analogous glycosylation decoys using thioglycosides are shown herein to decoy N-glycosylation that includes the inflammatory ligand Sialyl-Le^{x} (Sialyl Lewis X), that can mitigate the first steps of inflammation by neutrophils exhibiting this marker binding to E-selectin on capillary endothelium.

### SUMMARY

In accordance with the purpose(s) of the present disclosure, as embodied and broadly described herein, the disclosure, in one aspect, relates to glycosides comprising Formula I or Formula II:

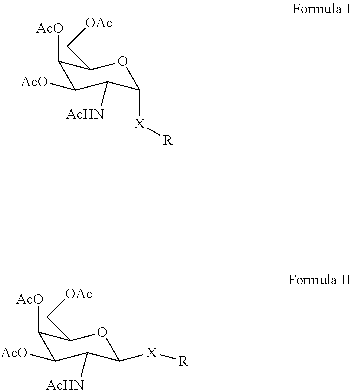

wherein X is selected from O, S, or in the present disclosure, Se;
and wherein R is selected from C1-C20 linear or branched alkyl, alkenyl, aryl, alkylaryl, or arylalkyl.

Also disclosed are methods of making the compounds, pharmaceutical compositions comprising the compounds, and methods of treating or preventing viral diseases and their symptoms in a subject using the same. In one aspect, the glycosides target host cell glycan decoration of viral proteins and do not cause the viruses to mutate. In another aspect, the glycosides and pharmaceutical compositions comprising the same can be applied topically for some viruses, such as, for example, HSV-1, HSV-2, and Herpes zoster, and thereby limit systemic side effects in the subject.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows a model of the SARS-CoV-2 spike S-glycoprotein trimer including extensive glycosylation.

FIG. 2 shows petri dishes in which a plaque assay for Herpes simplex 1 was conducted with various combinations of exemplary compounds. One combination, (H) N,O-tetraacetyl-GlcNAc-β-S-geranyl (TE3048) (acetylated TE3054), an N-linked decoy combined with α-benzyl GalNAc (BOG) an O-linked decoy, completely blocked infectivity. TE3054 (B) was also inhibitory.

FIG. 3 shows magnification of a typical area from the plates in FIG. 2 and control plates. In Panels D,E,F are 3 examples of massive plaque damage from Herpes Simplex 1 in untreated Vero cultures. In the first column are treatments with (A) 5 mg/mL benzyl-O-GalNac (BOG); (B) (2.5 mg BOG+0.5 mg GlcNAc-S-geranyl (TE3054) and (C) 0.2 mg/mL TE3054 alone. The combination showed synergistic inhibition (B). The combination of BOG and N,O-tetra-acetyl-GlcNAc-β-S-geranyl (TE3048) showed complete inhibition of Herpes Simplex 1 infection (G).

FIG. 4 shows a dose response curve for exemplary compound TE0348 alone.

FIGS. 5A-5B show exemplary compound JR002 significantly inhibits HSV-2 replication in human cells. FIG. 5A. Fluorescent images of MRC5 cells infected with HSV-2 GFP+(bHSV2-BAC38-S1). Images taken at 0 and 24 hours post infection using an Incucyte S3 Live-Cell Analysis System. Acyclovir at a matched concentration (125 μM) was used as a positive control. FIG. 5B. Live cell imaging analysis of triplicate wells infected with HSV-2 GFP+. Wells were treated with either Vehicle (0.1% DMSO), Acyclovir 125 μM, or JR002 125 μM. Images were collected at 2 hour intervals for 48 hours post infection. Relative fluorescent intensity (RFI) calculated as the percent increase in fluorescence intensity relative to time 0 post infection. Bars represent the standard error of the mean.

FIG. 6 shows that exemplary compound TE3048 exhibits 60-70% inhibition of SARS-CoV-2 at the highest non-toxic dose.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Disclosed herein is a novel approach to virus inhibition: targeting viruses with decoy glycosides, or host cell N-glycosylation and O-glycosylation systems that are hijacked by viruses to glycosylate coat and spike proteins. The disclosed decoy compounds are thioglycosides and selenoglycosides that target N- and O-glycosylation, which is key to trafficking glycoproteins through to proper folding and maturation, through the calnexin calreticulin chaperone system. Disrupting N-glycosylation machinery leads to slowing and stopped trafficking of spike proteins including initiating the unfolded protein response, crucial for viral infectivity and membrane fusion. The glycosylation machinery transfers sugars to the disclosed decoys, competing with the viral target.

A substrate decoy approach to disrupt viral glycoprotein maturation using thioglycosides and selenoglycosides requires typically higher concentrations to compete with natural substrates than drugs that act as enzyme inhibitors. Viruses with coat proteins, such as SARS-CoV-2, hijack the host cell system to glycosylate their spike proteins. Interference with host cell glycan decoration of viral proteins is a potential target for therapeutics. This is an orthogonal approach, not targeting the virus itself, but interdicting host glycosylation of viral proteins. This approach is pan-strain because the host cell glycosylation system is targeted instead of the virus which cannot mutate to avoid host processing interference.

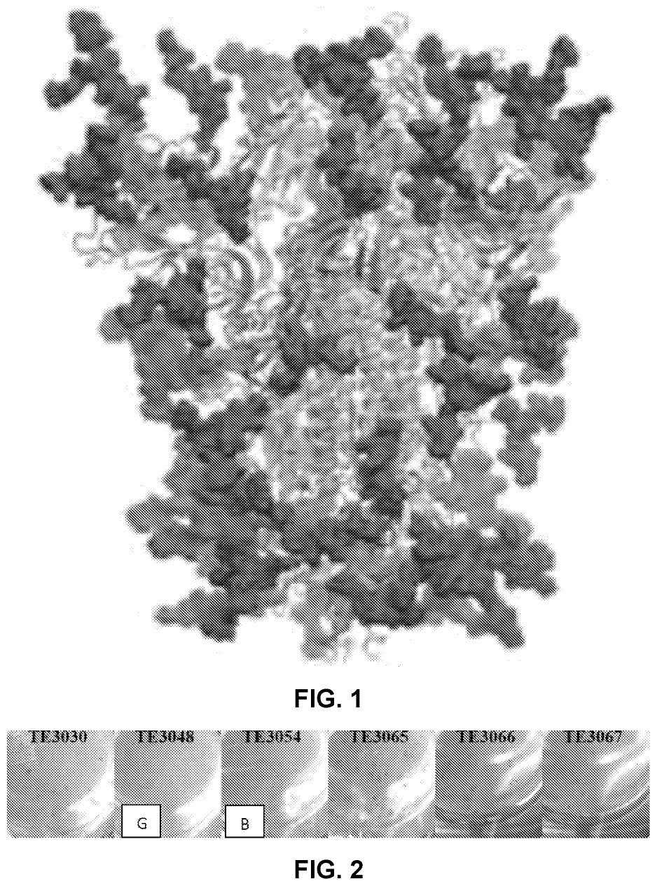

The S-glycoprotein of SARS-CoV-2 consists of a trimer, each of which has 18 N-glycosylation sites[(FIG. 1). The S-glycoprotein's function is to bind the ACE2 receptor on human cells for infectious entry. Also, host-specific glycans are recognized as “self” by the immune system, and confer S-protein protection, which in pauciglycosylated form will be more susceptible to antibodies and/or proteases, will not be properly folded, and may not be trafficked properly through the calnexin system to function in the infectivity cycle.

Preliminary viral interference data with the disclosed selenoglycoside N-linked glycosylation decoys against HSV-2, which has glycosylated coat proteins important in cell fusion, showed complete inhibition of infectivity in MRC5 cells, leading to the hypothesis that SARS-CoV-2 spike glycoproteins could be similarly affected.

The ACE2 receptor is heavily glycosylated with 7 N-glycans and potential O-glycans and its folding, trafficking, and function depend on proper glycosylation. Thioglycoside or selenoglycoside decoys can cause ACE2 to be defective in glycosylation and also to be affected in folding and trafficking. Therefore, host glycosylation of proteins is a therapeutic target for both the SARS-CoV-2 spike protein and the ACE2 receptor.

Thioglycoside and selenoglycoside decoys present a strong value proposition for COVID19 patients because as an adjunct treatment for infected patients, they may have an immediate effect to interrupt viral infection, so as not to progress to ARDS, while also having anti-inflammatory activity with minimized side effects.

A competitive advantage of thioglycoside and selenoglycoside decoys is their unique activities as antivirals that interfere with spike protein and ACE2 receptor processing. This approach may target a wide range of coronaviruses, RSV, and even influenza for future pandemics. Another competitive advantage is that thioglycoside and selenoglycosides are much less expensive to produce than vaccines or antibodies, being a small molecule synthetic product produced in 3-4 steps. Most of the current therapeutics are costly monoclonal antibodies that are labor-intensive to produce. Without wishing to be bound by theory, a third competitive advantage is that the host cell glycosylation machinery is the target, which the virus cannot mutate to generate drug resistance. Thus, in an aspect, the decoys should work regardless of viral strain or type. A fourth competitive advantage is that 3 inhibitors of N-glycosylation have already been approved by the FDA showing precedence for their relative safety and therapeutic window.

Sugar chains (glycans) assist in the normal conformation, trafficking through the calnexin calreticulin chaperone system and folding and maturation of the S19-glycoprotein, whose function is important to bind the ACE2 receptor on human cells for infectious entry. In concert, the ACE2 receptor is also heavily glycosylated, thereby presenting a second target for the decoys. Host-specific glycans on viral proteins are not targeted by the host immune system, instead protecting S-protein. In pauciglycosylated form, S19 is certain to be more susceptible to proteases, not be properly folded, may not function in the infectivity cycle, and may expose antigens for the adaptive immune system.

Since the target is the host glycosylation system, the virus cannot mutate to overcome this approach to pharmaceutical intervention. Tamiflu, for example, blocked a hemagglutination protein site on the influenza viral surface, and the virus mutated resistance. Similarly, vaccines target surface proteins on viruses, and mutation renders these ineffective, requiring new vaccines each year or each time a new variant emerges.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a cell line,” “a virus,” or “a glycoside,” includes, but is not limited to, mixtures or combinations of two or more such cell lines, viruses, or glycosidases, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less' and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a pharmaceutical composition refers to an amount that is sufficient to achieve the desired improvement in the property modulated by the formulation component, e.g. achieving the desired level of control of viral replication or virus symptoms. The specific level in terms of wt % in a composition required as an effective amount will depend upon a variety of factors including the age and weight of the subject, prior vaccination status, severity of the symptoms, and any preexisting conditions or comorbidities experienced by the subject.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### ASPECTS

The present disclosure can be described in accordance with the following numbered aspects, which should not be confused with the claims.

Aspect 1. A glycoside comprising Formula I or Formula II:

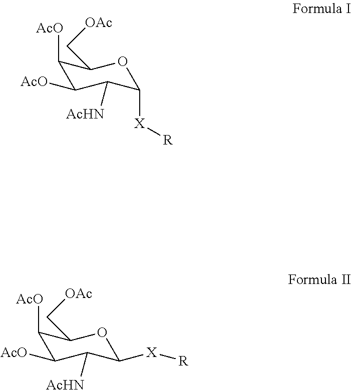

wherein X is selected from O, S, or Se;
and wherein R is selected from C1-C20 linear or branched alkyl, alkenyl, aryl, alkylaryl, or arylalkyl.

Aspect 2. The glycoside of aspect 1, wherein the glycoside has Formula I.

Aspect 3. The glycoside of aspect 1, wherein the glycoside has Formula II.

Aspect 4. The glycoside of any one of aspects 1-3, wherein X is O.

Aspect 5. The glycoside of any one of aspects 1-3, wherein X is S.

Aspect 6. The glycoside of any one of aspects 1-3, wherein X is Se.

Aspect 7. The glycoside of any one of aspects 1-3, wherein R is selected from geranyl, benzyl, methyl naphthyl, octyl, naphthyl, or any combination thereof.

Aspect 8. The glycoside of any one of aspects 1-4, wherein the glycoside is selected from N,O-tetra-acetyl-GlcNAc-β-S-geranyl, N,O-tetra-acetyl-GlcNAc-β-S-benzyl, N,O-tetra-acetyl-GlcNAc-β-S-octyl, N,O-tetra-acetyl-GlcNAc-β-S-naphthyl, N,O-tetra-acetyl-GlcNAc-β-S-methylnaphthyl, N,O-tetra-acetyl-GalNAc-α-O-benzyl, N,O-tetra-acetyl-GalNAc-α-O-methylnaphthyl, N,O-tetra-acetyl-GalNAc-α-S-benzyl, N,O-tetra-acetyl-GalNAc-α-S-methylnaphthyl, N,O-tetra-acetyl-GalNAc-α-S-geranyl, N,O-tetra-acetyl-GalNAc-α-S-octyl, N,O-tetra-acetyl-GalNAc-α-S-naphthyl, N,O-tetra-acetyl-GlcNAc-β-Se-geranyl, N,O-tetra-acetyl-GlcNAc-β-Se-methylnaphthyl, N,O-tetra-acetyl-GalNAc-α-Se-benzyl, N,O-tetra-acetyl-GalNAc-α-Se-methylnaphthyl, N,O-tetra-acetyl-GlcNAc-β-Se-naphthyl, N,O-tetra-acetyl-GlcNAc-β-Se-octyl, N,O-tetra-acetyl-GalNAc-α-Se-octyl, or any combination thereof.

Aspect 9. A pharmaceutical composition comprising the compound of any one of aspects 1-8.

Aspect 10. A method for treating or preventing at least one symptom of a virus in a subject, the method comprising administering the compound of any one of aspects 1-8 or the pharmaceutical composition of aspect 9 to the subject.

Aspect 11. The method of aspect 10, wherein the subject is a mammal.

Aspect 12. The method of aspect 11, wherein the mammal is a human, dog, cat, goat, sheep, horse, cattle, swine, hamster, rat, mouse, guinea pig, or rabbit.

Aspect 13. The method of any one of aspects 10-12, wherein the virus is a herpes virus, respiratory syncytial virus (RSV), an influenza virus, or a coronavirus.

Aspect 14. The method of aspect 13, wherein the herpes virus is HSV-1 or HSV-2.

Aspect 15. The method of aspect 13, wherein the coronavirus is SARS-CoV-2.

Aspect 16. The method of any one of aspects 10-15, wherein the compound or pharmaceutical composition is administered topically to at least one infected site.

Aspect 17. The method of any one of aspects 10-16, wherein the compound or pharmaceutical composition is formulated as a cream, gel, salve, or eye drop.

Aspect 18. The method of any one of aspects 10-17, wherein the method does not cause systemic side effects in the subject.

Aspect 19. The method of any one of aspects 10-18, wherein the glycoside interferes with host cell glycan decoration of viral proteins.

Aspect 20. The method of any one of aspects 10-19, wherein the method does not cause the virus to mutate.

### Examples

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Synthesis of Decoys

### α-Selenoqlycoside Synthesis

Synthesis of bis(2-naphthylmethyl) diselenide is presented in Scheme 1:

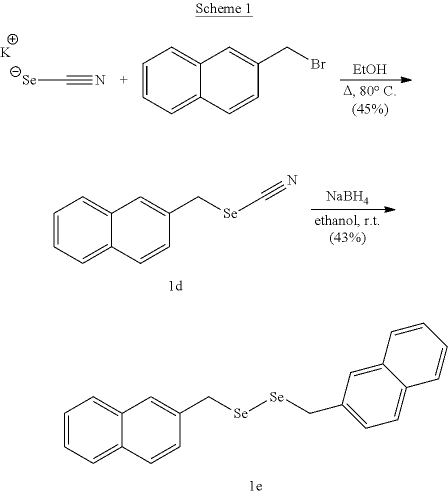

Synthesis of di-n-octyl diselenide is presented in Scheme 2:

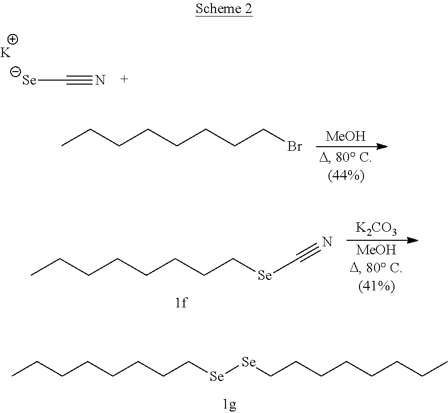

Synthesis of 2-acetamido-2-deoxy-3,4,6-tri-O-acetyl-1-seleno-α-D-galactopyranoside is presented in scheme 3:

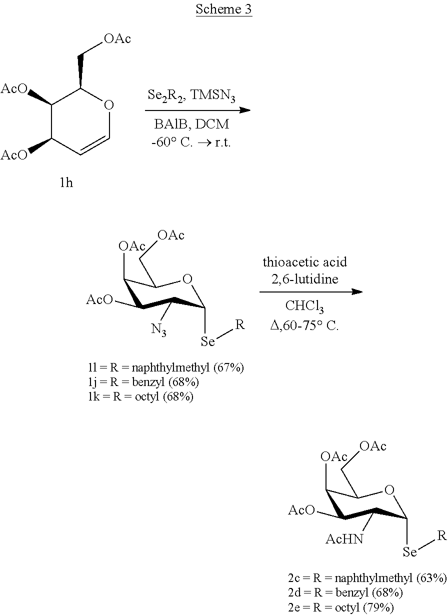

This reaction is conducted at room temperature (about 18-22° C.).

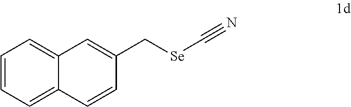 [SMILES: N#C[Se]Cc1ccc2ccccc2c1]

2-(selenocyanatomethyl)naphthalene (1d). Potassium selenocyanate (2.59 g, 18.0 mmol) and 2-(bromomethyl)naphthalene (3.14 g, 14.2 mmol) were dissolved in 15 mL of absolute ethanol. The reaction was heated in an oil bath preheated to 80° C. for 6 hours. After this time, the hot reaction mixture was immediately vacuum filtered to remove the inorganic salt (KBr). The resulting filtrate was left to cool at room temperature where 1d precipitated out upon cooling. The crystals were isolated via vacuum filtration to yield 1d as a yellow crystalline solid (1.57 g, 45%) requiring no further purification. ¹H NMR matched previously reported spectra.

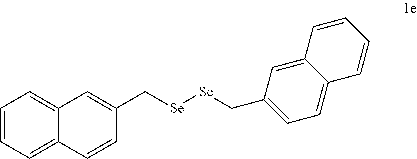 [SMILES: c1ccc2cc(C[Se][Se]Cc3ccc4ccccc4c3)ccc2c1]

Bis(2-naphthylmethyl) diselenide (1e). 1d (1.57 g, 6.39 mmol) was dissolved in 50 mL of absolute ethanol. Sodium borohydride (0.24 g, 6.4 mmol) was added carefully in small portions. The reaction was allowed to stir uncapped at room temperature for 2 hours. The solvent was removed under vacuum and the crude mixture was diluted with dichloromethane (1×30 mL) and washed with water (1×30 mL). The organic layer was collected, dried over sodium sulfate and concentrated to afford 1e as a yellow solid (1.21 g, 43%) requiring no further purification. ¹H NMR matched previously reported spectra.

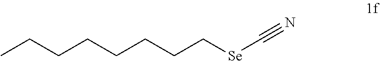

1-selenocyanatooctane (1f). Potassium selenocyanate (2.46 g, 17.1 mmol) and 1-bromooctane (2.7 mL, 15.5 mmol) were dissolved in 15 mL of methanol. The reaction was refluxed in an oil bath preheated to 80° C. for one hour. After this time, the hot reaction mixture was immediately vacuum filtered to remove the inorganic salt (KBr). The resulting filtrate was transferred to a separatory funnel and the aqueous layer was extracted with hexanes (2×50 mL). The combined organic layers were dried over sodium sulfate and concentrated. The crude reaction mixture (a loose flowing oil) was run through a short silica plug (with no use of solvent) to afford 1f as a yellow oil (1.48 g, 44%). ¹H NMR matched previously reported spectra.

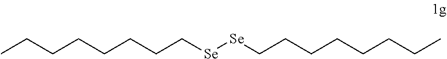 [SMILES: CCCCCCCC[Se][Se]CCCCCCCC]

Di-n-octyl diselenide (1g). 1f (1.48 g, 6.78 mmol) was dissolved in 10 mL of methanol. Potassium carbonate (0.94 g, 6.8 mmol) was added in one portion. The reaction was refluxed in an oil bath preheated to 80° C. for 2 hours. The reaction mixture was transferred to a separatory funnel and the aqueous layer was extracted with hexanes (2×50 mL). The combined organic layers were dried over sodium sulfate and concentrated. The crude reaction mixture (a loose flowing oil) was run through a short silica plug (with no use of solvent) to afford 1g as a yellow oil (1.06 g, 41%). ¹H NMR matched previously reported spectra.

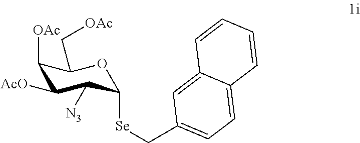

2-Azido-3,4,6-tri-O-acetyl-2-deoxy-1-seleno-naphthylmethyl-α-D-galactopyranoside (1i). Tri-O-acetyl-D-galactal 1h (0.49 g, 1.8 mmol) was dissolved in 10 mL of dichloromethane. The reaction mixture was placed in an acetonitrile and dry ice bath and cooled to −60° C. Bis(2-naphthylmethyl) diselenide (0.80 g, 1.8 mmol) and bis(acetoxy)iodobenzene 0.59 g, 1.8 mmol) were both added in one portion. Lastly, trimethylsilyl azide (0.47 mL, 3.6 mmol) was added dropwise using a syringe. The reaction was removed from the ice bath and gradually warmed to room temperature. The reaction was allowed to stir for 24 hours. After this time, the reaction was quenched with saturated sodium bicarbonate (1×20 mL) and transferred to a separatory funnel. The organic layer was collected, dried over sodium sulfate and concentrated. The crude reaction mixture was purified via silica gel flash column chromatography using a solvent gradient of 5-30% ethyl acetate in hexanes to afford 1i (0.65 g, 67%) as a yellow solid.

¹H NMR (500 MHz, CDC₃ with 0.03% TMS) δ 7.82-7.79 (m, 3H), 7.70 (s, 1H), 7.50-7.44 (m, 3H), 5.62 (d, J=5.7 Hz, 1H), 5.46-5.45 (m, 1H), 5.12 (dd, J=10.7 Hz, 3.3 Hz, 1H), 4.61 (t, J=6.4 Hz, 1H), 4.16 (d, J=6.5 Hz, 1H), 4.13-4.11 (m, 2H), 3.98 (d, J=12.1 Hz, 1H), 3.92 (d, J=12.1 Hz, 1H), 2.16 (s, 3H), 2.07 (s, 3H), 2.03 (s, 3H). ¹³C NMR (125 MHz, CDC₃ with 0.03% TMS) δ 170.4, 170.0, 169.6, 135.4, 133.2, 132.4, 128.6, 127.7, 127.6, 127.3, 127.2, 126.3, 125.9, 78.3, 71.6, 68.7, 67.2, 61.9, 58.3, 26.2, 20.7, 20.6, 20.6 HRMS (ESI): m/z [M+Na]+ calcd for C₂₃H₂₅N₃O₇SeNa 558.0755; found 558.0751. [α]_{D}²⁵=+110.0 (c 0.01, CH₂Cl₂). IR (cm⁻¹) 2108, 1742, 1367, 1211, 1048, 904, 818, 749, 545, 478.

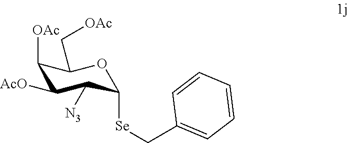

2-Azido-3,4,6-tri-O-acetyl-2-deoxy-1-seleno-benzyl-α-D-galactopyranoside (1j). Tri-O-acetyl-D-galactal 1h (0.51 g, 1.9 mmol) was dissolved in 10 mL of dichloromethane. The reaction mixture was placed in an acetonitrile and dry ice bath and cooled to −60° C. Dibenzyl diselenide⁵ (0.64 g, 1.9 mmol) and bis(acetoxy)iodobenzene (0.60 g, 1.9 mmol) were both added in one portion. Lastly, trimethylsilyl azide (0.49 mL, 3.7 mmol) was added dropwise using a syringe. The reaction was removed from the ice bath and gradually warmed to room temperature. The reaction was allowed to stir for 24 hours. After this time, the reaction was quenched with saturated sodium bicarbonate (1×10 mL) and transferred to a separatory funnel. The organic layer was collected, dried over sodium sulfate and concentrated. The crude reaction mixture was purified via silica gel flash column chromatography using a solvent gradient of 5-30% ethyl acetate in hexanes to afford 1j (0.64 g, 68%) as a yellow solid.

¹H NMR (500 MHz, CDC₃ with 0.03% TMS) δ 7.31-7.30 (m, 4H), 7.25-7.21 (m, 1H), 5.65 (d, J=7.0 Hz, 1H), 5.45-5.44 (m, 1H), 5.11 (dd, J=13.4 Hz, 4.1 Hz, 1H), 4.57 (t, J=8.6 Hz, 1H), 4.18-4.12 (m, 3H), 3.83-3.73 (m, 2H), 2.16 (s, 3H), 2.07 (s, 3H), 2.03 (s, 3H). ¹³C NMR (125 MHz, CDC₃ with 0.03% TMS) δ 170.3, 169.9, 169.6, 138.2, 128.9, 128.6, 127.0, 78.3, 71.5, 68.7, 67.2, 61.8, 58.4, 25.9, 20.7, 20.6, 20.6. HRMS (ESI): m/z [M+Na]+ calcd for C₁₉H₂₃N₃O₇SeNa 508.0587; found 508.0589. [α]_{D}²⁵=+248.1 (c 0.01, CH₂Cl₂). IR (cm⁻¹) 2109, 1744, 1367, 1211, 1029, 942, 905, 758, 696, 608, 548, 441.

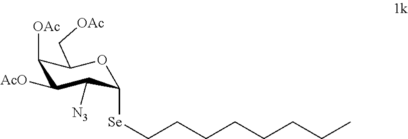

2-Azido-3,4,6-tri-O-acetyl-2-deoxy-1-seleno-octyl-α-D-galactopyranoside (1k). Tri-O-acetyl-D-galactal 1h (0.38 g, 1.4 mmol) was dissolved in 10 mL of dichloromethane. The reaction mixture was placed in an acetonitrile and dry ice bath and cooled to −60° C. Di-n-octyl diselenide (0.54 g, 1.4 mmol) and bis(acetoxy)iodobenzene (0.45 g, 1.4 mmol) were both added in one portion. Lastly, trimethylsilyl azide (0.37 mL, 2.8 mmol) was added dropwise using a syringe. The reaction was removed from the ice bath and gradually warmed to room temperature. The reaction was allowed to stir for 24 hours. After this time, the reaction was quenched with saturated sodium bicarbonate (1×20 mL) and transferred to a separatory funnel. The organic layer was collected, dried over sodium sulfate, and concentrated. The crude reaction mixture was purified via silica gel flash column chromatography using a solvent gradient of 5-30% ethyl acetate in hexanes to afford 1i (0.48 g, 68%) as a yellow oil.

¹H NMR (500 MHz, CDCl₃ with 0.03% TMS) δ 5.84 (d, J=6.9 Hz, 1H), 5.44-5.43 (m, 1H), 5.12 (dd, J=13.5 Hz, 4.2 Hz, 1H), 4.56-4.52 (m, 1H), 4.21-4.17 (m, 1H), 4.12-4.11 (m, 2H), 2.71-2.64 (m, 1H), 2.59-2.53 (m, 1H), 2.16 (s, 3H), 2.05 (m, 6H), 1.74 (m, 4H), 1.38-1.25 (m, 8H), 0.90-0.87 (m, 3H). ¹³C NMR (125 MHz, CDCl₃ with 0.03% TMS) δ 170.3, 170.0, 169.6, 79.1, 71.2, 68.5, 67.3, 61.7, 58.6, 31.8, 30.2, 30.0, 29.2, 29.1, 23.5, 22.6, 20.7, 20.6, 20.6, 14.1. HRMS (ESI): m/z [M+] calcd for C₂₀H₃₃N₃O₇Se 507.1484 found 507.1430. [α]_{D}²⁵=+134.9 (c 0.01, CH₂Cl₂). IR (cm⁻¹) 2108, 1746, 1368, 1212, 1079, 1049, 942, 906, 715, 651, 601, 546, 482, 420.

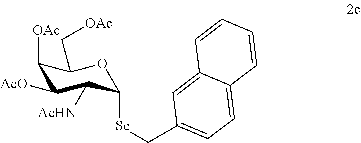

2-Acetamido-2-deoxy-3,4,6-tri-O-acetyl-1-seleno-naphthylmethyl-α-D-glucopyranoside (2c). 1i (0.65 g, 1.2 mmol) was dissolved in 4 mL of chloroform. 2,6-lutidine (0.36 mL, 3.1 mmol) was added in one portion with a syringe. Lastly, thioacetic acid (0.22 mL, 3.1 mmol) was added dropwise. The reaction was heated in an oil bath preheated to 60° C. for 36 hours (the oil bath temperature fluctuated between 60-75° C. during the reaction). After this time, the solvent was concentrated and the crude reaction mixture was purified via silica gel flash column chromatography using a solvent gradient of 10-35% ethyl acetate in hexanes to afford 2c (0.42 g, 63%) as a white solid.

¹H NMR (500 MHz, CDCl₃ with 0.03% TMS) δ 7.80-7.77 (m, 2H), 7.69 (s, 1H), 7.49-7.41 (m, 4H), 5.84 (d, J=5.2 Hz, 1H), 5.40-5.39 (m, 2H), 5.02 (dd, J=11.5 Hz, 3.3 Hz, 1H), 4.67-4.62 (m, 1H), 4.53 (t, J=6.6 Hz, 1H), 4.18 (dd, J=11.3 Hz, 6.1 Hz, 1H), 4.09 (d, J=6.9 Hz, 1H), 4.06 (t, J=3.7 Hz, 1H), 4.03 (m, 1H), 2.15 (s, 3H), 2.04 (s, 3H), 1.99 (s, 3H), 1.76 (s, 3H). ¹³C NMR (125 MHz, CDCl₃ with 0.03% TMS) δ 171.1, 170.4, 170.2, 170.0, 135.6, 133.3, 132.5, 128.6, 127.7, 127.2, 126.9, 126.4, 126.0, 82.5, 69.5, 69.5, 67.1, 61.8, 48.8, 27.7, 23.1, 20.7, 20.7, 20.7. HRMS (ESI): m/z [M+Na]+ calcd for C₂₅H₂₉NO₈SeNa 574.0956; found 574.0954; m/z [M+K]+calcd for C₂₅H₂₉NO₈SeK 590.0695; found 590.0698. [α]_{D}²⁵=+266.6 (c 0.01, CH₂Cl₂). IR (cm⁻¹) 1742, 1657, 1538, 1368, 1215, 1046, 909, 818, 750, 572, 463.

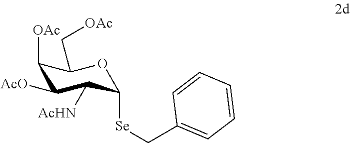

2-Acetamido-2-deoxy-3,4,6-tri-O-acetyl-1-seleno-benzyl-α-D-glucopyranoside (2d). 1j (0.64 g, 1.3 mmol) was dissolved in 4 mL of chloroform. 2,6-lutidine (0.40 mL, 3.4 mmol) was added in one portion with a syringe. Lastly, thioacetic acid (0.24 mL, 3.4 mmol) was added dropwise and the reaction was heated in an oil bath preheated to 60° C. for 36 hours (the oil bath temperature fluctuated between 60-75° C. during the reaction). After this time, the solvent was concentrated and the crude reaction mixture was purified via silica gel flash column chromatography using a solvent gradient of 10-35% ethyl acetate in hexanes to afford 2d (0.41 g, 63%) as a pale pink solid.

¹H NMR (500 MHz, CDC₃ with 0.03% TMS) δ 7.30-7.28 (m, 4H), 7.24-7.21 (m, 1H), 5.81 (d, J=5.2 Hz, 1H), 5.43 (d, J=8.6 Hz, 1H), 5.40-5.39 (m, 1H), 5.01 (dd, J=11.6 Hz, 3.3 Hz, 1H), 4.67-4.62 (m, 1H), 4.52-4.49 (m, 1H), 4.17 (dd, J=11.4 Hz, 6.2 Hz, 1H), 4.07 (dd, J=11.3 Hz, 6.9 Hz, 1H), 3.89-3.83 (m, 2H), 2.15 (s, 3H), 2.05 (s, 3H), 1.99 (s, 3H), 1.84 (s, 3H). ¹³C NMR (125 MHz, CDCl₃ with 0.03% TMS) δ 171.1, 170.4, 170.2, 170.1, 138.3, 128.8, 128.7, 127.2, 82.3, 69.5, 69.4, 67.1, 61.8, 48.8, 27.3, 23.2, 20.7, 20.7. HRMS (ESI): m/z [M+Na]+ calcd for C₂₁H₂₇NO₈SeNa 524.0859; found 524.0876. [α]_{D}²⁵=+217.2 (c 0.01, CH₂Cl₂). IR (cm⁻¹) 1743, 1657, 1522, 1369, 1216, 1047, 911, 697, 573, 471.

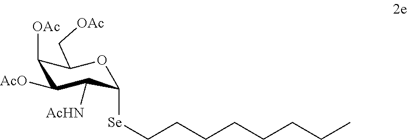

2-Acetamido-2-deoxy-3,4,6-tri-O-acetyl-1-seleno-octyl-α-D-glucopyranoside (2e). 1k (0.48 g, 0.95 mmol) was dissolved in 3 mL of chloroform. 2,6-lutidine (0.29 mL, 2.5 mmol) was added in one portion with a syringe. Lastly, thioacetic acid (0.18 mL, 2.6 mmol) was added dropwise and the reaction was heated in an oil bath preheated to 60° C. for 36 hours (the oil bath temperature fluctuated between 60-75° C. during the reaction). After this time, the solvent was concentrated and the crude reaction mixture was purified via silica gel flash column chromatography using a solvent gradient of 10-35% ethyl acetate in hexanes to afford 2e (0.39 g, 79%) as a brown oil.

¹H NMR (500 MHz, CDC₃ with 0.03% TMS) δ 5.86 (d, J=5.2 Hz, 1H), 5.59 (d, J=8.6 Hz, 1H), 5.39-5.38 (m, 1H), 5.02 (dd, J=11.5 Hz, J=3.3 Hz, 1H), 4.69-4.64 (m, 1H), 4.52-4.49 (m, 1H), 4.18-4.12 (m, 1H), 4.10-4.06 (m, 1H), 2.73-2.67 (m, 1H), 2.65-2.60 (m, 1H), 2.16 (s, 3H), 2.05 (s, 3H), 2.01 (s, 3H), 1.97 (s, 3H), 1.71-1.65 (m, 4H), 1.38-1.25 (m, 8H), 0.90-0.87 (m, 3H). ¹³C NMR (125 MHz, CDC₃ with 0.03% TMS) δ 171.1, 170.4, 170.3, 170.2, 82.7, 69.4, 69.1, 67.1, 61.8, 48.9, 31.8, 30.6, 29.9, 29.2, 29.0, 24.9, 23.3, 22.6, 20.8, 20.7, 20.7, 14.1. HRMS (ESI): m/z [M+Na]+C₂₂H₃₇NO₈SeNa calcd for 546.1578; found 546.1579. [α]_{D}²⁵=+63.2 (c 0.01, CH₂Cl₂). IR (cm⁻¹) 1746, 1657, 1541, 1369, 1215, 1116, 1082, 1047, 912, 720, 589, 573, 547, 473, 411.

### β-Selenoqlycoside Synthesis

Synthesis of 2-acetamido-2-deoxy-3,4,6-tri-O-acetyl-1-seleno-β-D-glucopyranoside is presented in Scheme 4:

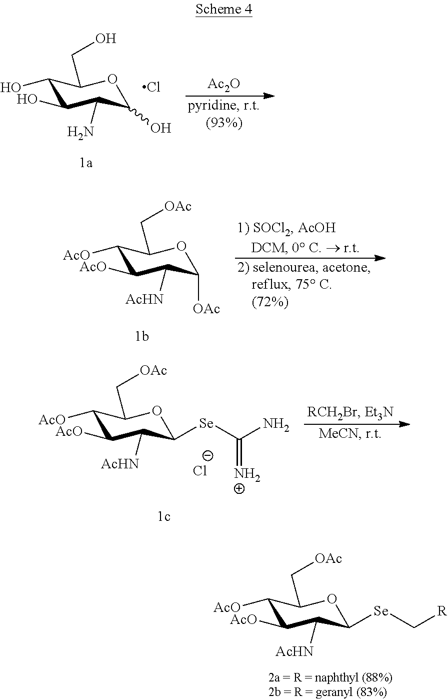

This reaction is conducted at room temperature (about 18-22° C.).

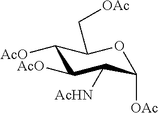

2-Acetamido-1,3,4,6-tetra-O-acetyl-2-deoxy-D-glucopyranose (1b). Glucosamine hydrochloride (5.00 g, 23.2 mmol), 1a, was dissolved in pyridine (20 mL) and treated with acetic anhydride (32.9 mL, 347 mmol) at room temperature. The reaction stirred overnight (18 h), and reaction completion was confirmed via thin layer chromatography¹. The solvent was removed under vacuum, and the residue was diluted with dichloromethane (100 mL) and washed with water (3×100 mL) and saturated brine (1×100 mL). The combined organic layers were dried over sodium sulfate and concentrated to afford intermediate 1b as a white solid requiring no further purification (8.41 g, 93%). ¹H NMR matched previously reported spectra.

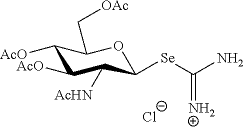

2-Acetamido-2-deoxy-3,4,6-tri-O-acetyl-β-D-glucopyranosylisoselenuronium chloride (1c). A solution of 1b (5.00 g, 12.8 mmol) in dichloromethane (20 mL) was cooled to 0° C. and treated with acetic acid (2.48 mL, 43.7 mmol) and thionyl chloride (15.1 mL, 208 mmol). The reaction gradually warmed to room temperature and stirred overnight (20 h)³. The reaction was then cooled to 0° C. and slowly quenched using a cold, saturated solution of sodium bicarbonate (100 mL). The crude mixture was transferred to a separatory funnel and the organic layer was collected and washed with water (3×100 mL). The organic layer was dried over sodium sulfate and concentrated to afford the glucosyl chloride intermediate (3.78 g, 10.3 mmol) as a white solid requiring no further purification. Selenourea (1.27 g, 10.3 mmol) was added to a solution of the glucosyl chloride (3.78 g, 10.3 mmol) in acetone (20 mL) and refluxed in an oil bath preheated to 75° C. for 40 minutes under inert atmosphere⁴. The reaction mixture was cooled to room temperature and the crude mixture was vacuum filtered and washed with acetone. The isolated solid was dried on high vacuum to afford the selenuronium chloride salt 1c (3.63 g, 72%) as a white solid requiring no further purification. ¹H NMR matched previously reported spectra.

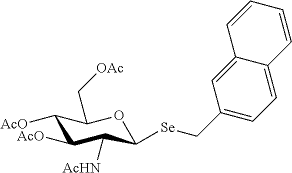

2-Naphthyl 2-acetamido-2-deoxy-3,4,6-tri-O-acetyl-1-seleno-3-D-glucopyranoside (2a). A solution of 1c (1.05 g, 2.15 mmol) in acetonitrile (10 mL) was treated with triethylamine (0.33 mL, 2.4 mmol) and 2-(bromomethyl)naphthalene (475 mg, 2.15 mmol) at room temperature. The reaction stirred overnight (14 h) at room temperature, and reaction completion was confirmed via thin layer chromatography. The solvent was removed under vacuum, and the residue was diluted with dichloromethane (30 mL) and washed with water (1×30 mL). The organic layer was dried over sodium sulfate and concentrated to yield the crude mixture as a dark gray solid. Silica gel flash column chromatography with a solvent gradient of 20-50% ethyl acetate in hexanes afforded 2a as a white solid (1.04 g, 88%).

¹H NMR (500 MHz, CDC₃ with 0.03% TMS) δ 7.84-7.77 (m, 3H), 7.69 (s, 1H), 7.51-7.45 (m, 3H), 5.25 (d, J=5.0 Hz, 1H), 5.12 (t, J=10.0, 1H), 4.92 (t, J=10.0, 1H), 4.42 (d, J=10.0 Hz, 1H), 4.34 (t, J=10.0, 1H), 4.27 (dd, J=5.0 Hz, 5.0 Hz, 1H), 4.22 (d, J=10.0 Hz, 1H), 4.19 (dd, J=2.9 Hz, 2.5 Hz, 1H), 4.04 (d, J=15.0 Hz, 1H), 3.55-3.51 (m, 1H), 2.14 (s, 3H), 2.01 (s, 3H), 1.99 (s, 3H), 1.87 (s, 3H). ¹³C NMR (125 MHz, CDC₃ with 0.03% TMS) δ 171.3, 170.7, 170.0, 169.2, 135.7, 133.3, 132.4, 128.5, 127.8, 127.5, 127.4, 127.2, 126.4, 126.0, 78.1, 73.9, 68.4, 62.6, 53.5, 26.9, 23.2, 20.9 20.6, 20.6. HRMS (ESI) m/z: [M+H]+ Calcd for C₂₅H₃₀NO₈Se 552.11312; found 552.11214. [α]_{D}²⁵=−102.4° (c=0.01, CH₂Cl₂). IR (cm⁻¹) 1742, 1656, 1522, 1368, 1225, 1035, 909, 815, 744, 590, 463.

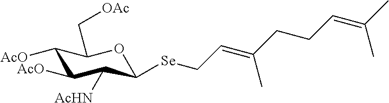

Geranyl 2-acetamido-2-deoxy-3,4,6-tri-O-acetyl-1-seleno-3-D-glucopyranoside (2b). A solution of 1c (1.05 g, 2.15 mmol) in acetonitrile (10 mL) was treated with triethylamine (0.33 mL, 2.4 mmol) and geranyl bromide (0.43 mL, 2.2 mmol) at room temperature. The reaction stirred overnight (14 h), and reaction completion was confirmed via thin layer chromatography. The solvent was removed under vacuum, and the residue was diluted with dichloromethane (30 mL) and washed with water (1×30 mL). The organic layer was dried over sodium sulfate and concentrated to yield the crude mixture as a dark gray oil. Silica gel flash column chromatography with a solvent gradient of 20-50% ethyl acetate in hexanes afforded 2b as a white solid (0.97 g, 83%). [0092]¹H NMR (500 MHz, CDC₃ with 0.03% TMS) δ 5.38-5.31 (m, 2H), 5.14-5.05 (m, 3H), 5.03-4.68 (m, 1H), 4.28-4.22 (m, 2H), 4.20-4.12 (m, 1H), 3.60-3.55 (m, 2H), 3.30-3.25 (m, 1H), 2.08-207 (m, 6H), 2.03 (s, 3H), 2.03 (s, 3H), 1.95 (s, 3H), 1.78-1.72 (m, 1H), 1.70-1.67 (m, 6H), 1.61-1.57 (m, 3H). ¹³C NMR (125 MHz, CDC₃ with 0.03% TMS) δ 171.3, 170.7, 169.9, 169.2, 139.5, 131.8, 123.9, 120.1, 78.5, 74.0, 68.3, 62.4, 53.7, 39.6, 26.6, 25.8, 23.3, 20.8, 20.7, 20.6, 17.8, 15.7. HRMS (ESI-TOF) m/z: [M+H]+ Calcd for C₂₄H₃₈NO₈Se 548.17572; found 548.17689. [α]_{D}²⁵=−45.9° (c=0.01, CH₂Cl₂). IR (cm⁻¹) 2925, 1740, 1658, 1535, 1370, 1221, 1039, 908, 596.

Thioglycosides, O-glycosides, and selenoglycosides have been or will be synthesized including at least the following. Thioglycosides and O-glycosides: GlcNAc-ß-S-geranyl (N-linked glycosylation decoy) (TE3054), not O-acetylated; N,O-tetra-acetyl-GlcNAc-ß-S-geranyl (N-linked glycosylation decoy) (TE3048); N,O-tetra-acetyl-GlcNAc-ß-S-benzyl (N-linked glycosylation decoy); N,O-tetra-acetyl-GlcNAc-ß-S-octyl (N-linked glycosylation decoy); N,O-tetra-acetyl-GlcNAc-ß-S-naphthyl (N-linked glycosylation decoy); N,O-tetra-acetyl-GlcNAc-ß-S-methylnaphthyl (N-linked glycosylation decoy); N,O-tetra-acetyl-GalNAc-α-O-benzyl (BOG) (0-linked glycosylation decoy); N,O-tetra-acetyl-GalNAc-α-O-methylnaphthyl (O-linked glycosylation decoy); N,O-tetra-acetyl-GalNAc-α-S-benzyl (O-linked glycosylation decoy); N,O-tetra-acetyl-GalNAc-α-S-methylnaphthyl (O-linked glycosylation decoy); N,O-tetra-acetyl-GalNAc-α-S-geranyl (O-linked glycosylation decoy); N,O-tetra-acetyl-GalNAc-α-S-octyl (O-linked glycosylation decoy); and N,O-tetra-acetyl-GalNAc-α-S-naphthyl (O-linked glycosylation decoy). Selenoglycosides: N,O-tetra-acetyl-GlcNAc-ß-Se-geranyl (N-linked glycosylation decoy); N,O-tetra-acetyl-GlcNAc-ß-Se-methylnaphthyl (N-linked glycosylation decoy); N,O-tetra-acetyl-GalNAc-α-Se-benzyl (O-linked glycosylation decoy); N,O-tetra-acetyl-GalNAc-α-Se-methylnaphthyl (O-linked glycosylation decoy); N,O-tetra-acetyl-GlcNAc-ß-Se-naphthyl (N-linked glycosylation decoy); N,O-tetra-acetyl-GlcNAc-ß-Se-octyl (N-linked glycosylation decoy); and N,O-tetra-acetyl-GalNAc-α-Se-octyl (O-linked glycosylation decoy).

### Example 2: In Vitro Testing

Thioglycoside N-glycosylation decoys and recently synthesized novel selenoglycoside N- and O-glycosylation decoys show potent antiviral activity. These compounds will be tested and optimized to inhibit infectivity of herpes simplex 1 and 2 (HSV-1 and HSV-2, respectively) as well as pseudotype VSV-S19 displaying the spike protein of SARS-CoV-2. The glycosides will be tested in primate cells including Vero, MRC5 and cells expressing hACE2 for inhibition of infectivity. N-glycosylation decoys disrupt host processing of viral coat proteins and hACE2 receptor proteins by administration in concentrations that compete for enzymes that normally transfer sugars to glycoproteins. This results in oligosaccharides attached to the decoys instead of the viral glycoprotein rendering the viral proteins pauciglycosylated and unlikely to be processed, folded properly, or trafficked through the calnexin calreticulin system. This glycosylation decoy approach has been validated in an anti-inflammatory system and the mechanism defined by inhibiting E-selectin ligand expression in leucocytes while transferring sugars to the decoys. Inhibition of N-glycan maturation and infectivity on the SARS-CoV-2 spike protein has previously been demonstrated by using the mannosidase inhibitor kifunensine that blocks N-glycan processing at the oligomannose stage. Zhao, et al. showed that inhibition of ER glucosidases prevented processing and folding of ACE2 with N-linked glycans impairing SARS1 and human coronavirus NL63 Spike protein mediated cell entry by preventing ACE2 glycan processing. Preliminary evidence of inhibition of viral replication has been observed in a re-infectivity plaque assay with HSV-1 on a primate cell line. Green Monkey Kidney Vero cells were infected with low multiplicity HSV-1 was plaque-free after treatment with a combination of tetra N,O-acetyl GlcNAc-β-thiogeranyl and GalNAc-α-O-benzyl, without overt toxicity to the cell layer (data shown herein) as measured by trypan blue exclusion. The disclosed selenoglycosides were preliminarily tested on HSV-2 in MRC5 cell host system and showed complete inhibition of infectivity at concentrations comparable to acyclovir that do not show toxicity.

Combinations of N- and O-linked glycosylation inhibitors will be tested to determine effects on HSV-1 and HSV-2 infectivity. Several thioglycoside and selenoglycoside decoys will further be tested in an S19 spike protein induced cell fusion model. The thioglycoside and selenoglycoside decoys will further be tested in a VSV S19 pseudotype cell binding assay. The effects of thioglycoside and selenoglycosides on spike protein binding to cells expressing hACE2 will further be tested and LD₅₀, MTD, toxicity, and optimum effective dose tests for thioglycoside and selenoglycoside decoys and combinations thereof on Vero and MRC5 cells will be determined.

An ocular Herpes 1 model in mice will be evaluated using the glycosides as a topical application in eyedrops to inhibit infection, addressing an orphan disease in humans in which about 60,000 cases occur per year. Topical application is expected to minimize systemic effects.

Thioglycosides and selenoglycosides will be tested in Vero and MRC5 cell viral infectivity models. Primate cell models of HSV-1, HSV-2, and SARS COV2 VSV-S19 pseudovirus, will be used in assays including assessment of cell fusion. infectivity, and functionality of the defective S-protein and ACE2 receptor. Thioglycosides and selenoglycosides will be tested in cell lines transformed with hACE2, using nonpathogenic VSV-S19 pseudotype for binding, plaque assays, fusion, replication.

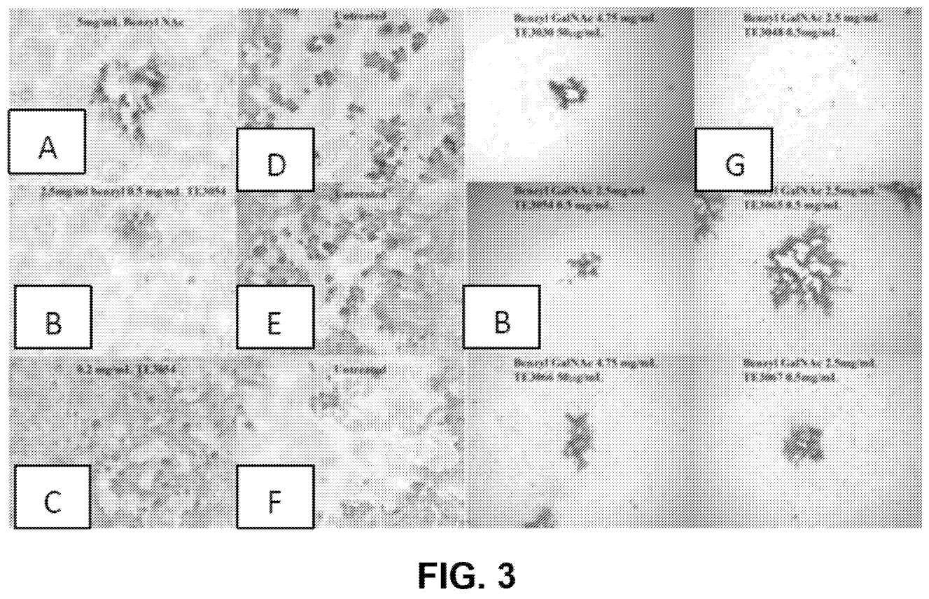

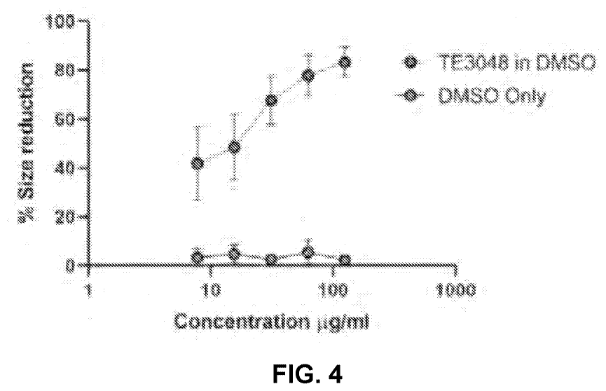

Preliminary work on viral inhibition by thioglycosides, shows that several compounds inhibit plaque formation of HSV-1 in a Vero primate cell line (FIG. 2). Solutions of thioglycoside test compounds were overlayed on the Vero cell layer in a 4 hour treatment. Low multiplicity HSV-1 was overlayed, to test infectivity by plaque formation (killed cells). In FIG. 3, TE3054 (GlcNAc-S-geranyl) was tested at 200 μg/mL (C), α-benzyl-GalNAc at 5 mg/mL (A) and a combination of the two (B), untreated controls on the right. The combination B was more effective than either compound alone. Results from this led to testing combinations of the O-linked and N-linked decoys shown in FIG. 3.

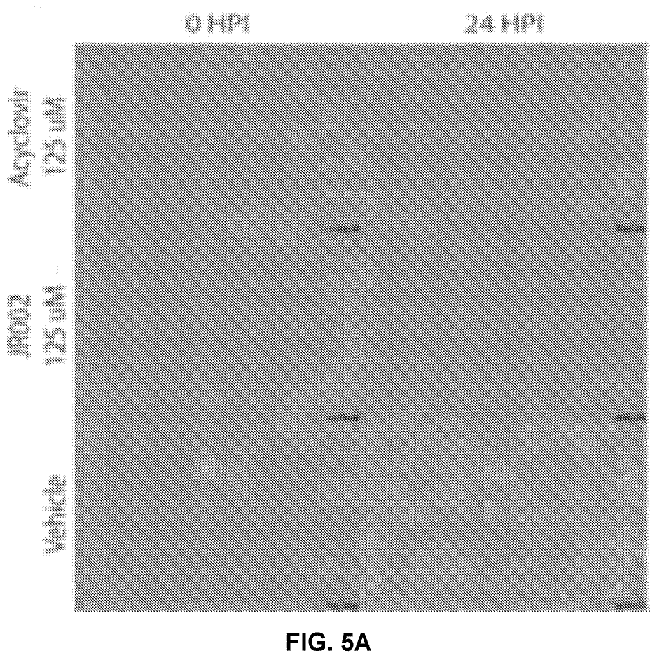

FIG. 4 shows a dose-response curve for TE3048 alone vs plaque size. These results predict potent antiviral activity by thioglycoside decoy interference with viral coat protein glycosylation, resulting in defective viral proteins.

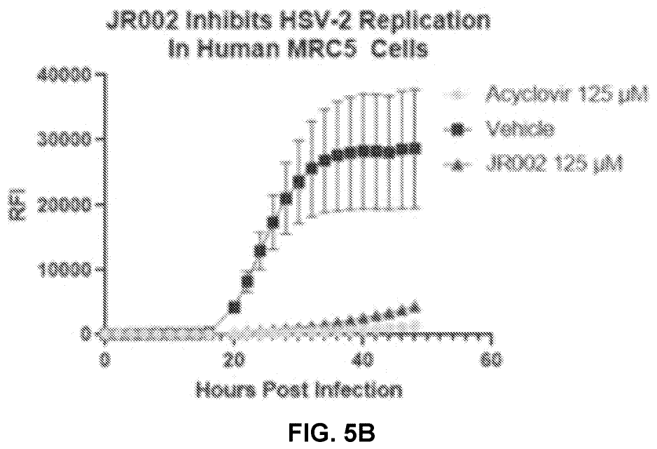

FIGS. 5A-5B show selenoglycoside JR002 (tetraacetyl-GlcNAc-β-Se-geranyl) inhibition of HSV-2 in human MRC5 cells: This preliminary experiment showed potent inhibition of infectivity for HSV-2 in MRC5 cells by the N-linked glycosylation decoy N,O-tetraacetyl-GlcNAc-β-Se-geranyl. JR002 is the selenoglycoside homolog of TE3048, the thioglycoside for which data for inhibition of HSV-1 was shown above. In FIG. 5A., the lower right quadrant represents high level cell damage, fluorescent HSV-2 GFP+ infected cells 24 hours post infection (24HPI), vehicle only. Upper and middle quadrants show almost complete inhibition by Acyclovir and JR002, respectively. FIG. 5B shows live cell imaging analysis of inhibition of infectivity of HSV-2 by acyclovir and JR002.

Cell fusion assays: The coronavirus spike protein is a class I fusion protein, and methods to be used are described in detail in the literature. Transient expression of the SARS-CoV-2 S glycoprotein in Vero cells caused extensive cell fusion (formation of syncytia). The SARS-CoV-2 spike expression plasmid used in this study was conducted as follows: S genes were placed under the control of the huma n cytomegalovirus (CMV) immediate early promoter and were engineered to contain either 3XFLAG or N-MYC epitope tags at their amino terminal ends. The S gene was cloned into p3XFLAGCMV9 (Sigma, MO) and pCMV3-SP-N-MYC (Sino Biological, PA) parental vector plasmids. The S1 subunit of the expression construct contained the same amino terminus up to aa700 (Gly). The N-terminal domain of the S2 subunit was engineered to be exactly as in S1 containing the N-MYC tag at its amino terminus and encompassing the S2 amino acid sequence 701-1273. Vero cells are grown on 24 well plates and transiently transfected with pCMV3-SP-N-MYC using lipofectamine 2000. 2 μL of lipofectamine and 0.5 μg of plasmid DNA are used for transfection of Vero cells. Following 48 hours, plates are examined by phase contrast microscopy for fused cells and images taken under live conditions, as well as either after formalin or methanol fixation. This model is available for testing thioglycoside and selenoglycoside interference with cell fusion induced by S-glycoprotein. The second test model is a cell line transformed with hACE2. Cells can be treated with S- and/or Se-glycosides prior to mock infectivity studies with VSV-S19 pseudotyped virus to examine S- and/or Se-glycosides' effect on the amount of competent hACE2 receptor that can bind virus. Mab against hACE2 receptor can be used for quantitation of the cell surface expression. In controls, allowing VSV-s19 to infect untreated cells can be followed by S- and/or Se-glycoside treatment to measure mitigation of infectivity within the cell layer.

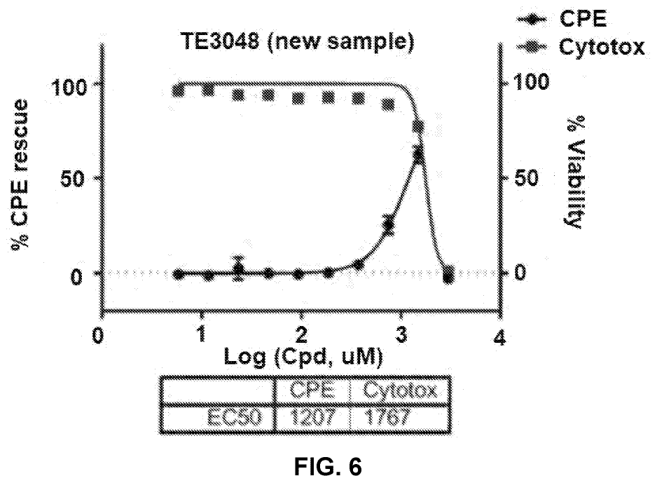

CPE Assay results are shown in FIG. 6; this represents a live SARS-CoV-2 virus assay measuring the ability of compounds to reverse the viral induced cytopathic effect (CPE) in Vero E6 host cells. In this assay, viral infection and replication leads to a loss of host cell viability, which is indirectly measured by an endpoint assessment of host cell viability after 72 hr. The CPE reduction assay indirectly monitors the ability of compounds to inhibit viral replication and infection through various molecular mechanisms, including direct inhibition of viral entry or enzymatic processes as well as acting on host pathways that modulate viral replication. Compounds with antiviral activity protect the host cells from the CPE of the virus, increasing viability.

Protocol: Vero E6 cells were premixed with SARS-CoV-2 for 5-10 min, dispensed into assay ready plates (pre-dispensed with compounds and controls). Cells and virus were incubated with compounds for 72 hr, then viability was assayed by Vero E6 host cell ATP content. 60 nL/well of compounds in DMSO was spotted into 384-well assay plates (Greiner, black clear bottom, tissue culture treated plates, #781091) by acoustic dispensing. In addition, 60 nL of the positive control Calpain inhibitor IV (stock concentration 2 mg/mL). 5 μL/well of media was dispensed into assay plates (MEM, 1% Pen/Strep/GlutaMax, 1% HEPES, 2% HI FBS). Dispensed 25 μL/well of Vero E6 cells inoculated with SARS CoV-2 (USA_WA1/2020) at multiplicity of infection (MOI) of 0.002 suspended in media. Final cell density was 4000 cells/well. Assay plates were incubated for 72 hr at 37° C., 5% CO₂, 90% humidity. Dispense 30 μL/well of CellTiter-Glo (Promega, Cat #G7573). Plates were Incubated for 10 min at room temperature.. Luminescence signal was read on Perkin Elmer Envision or BMG CLARIOstar plate reader.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Aguilera, B.; et al. Use of Cyclic Sulfamidates Derived from D-Allosamine in Nucleophilic Displacements: Scope and Limitations. Tetrahedron. 1997, 53, 5863-5876.
2. Bosch, B. J., et al., The coronavirus spike protein is a class I virus fusion protein: structural and functional characterization of the fusion core complex. J Virol, 2003. 77(16): p. 8801-11.
3. Cao, Z.; et al. Stereoselective Synthesis of Quercetin 3-O-Glycosides of 2-Amino-2-Deoxy-D-Glucose Under Phase Transfer Catalytic Conditions. Journal of Carbohydrate Chemistry. 2015, 34, 28-40.
4. Chatterjee, D.; et al. Cu(ClO₄)₂·6H₂O Catalyzed Solvent Free Per-O-Acetylation and Sequential One-Pot Conversions of Sugars to Thioglycosides. RCS Advances. 2015, 5, 29669-29674.
5. Guo, L., et al., Engineered trimeric ACE2 binds viral spike protein and locks it in “Three-up” conformation to potently inhibit SARS-CoV-2 infection. Cell Res, 2021. 31(1): p. 98-100.
6. Hoffmann, M., et al., SARS-CoV-2 Cell Entry Depends on ACE2 and TMPRSS2 and Is Blocked by a Clinically Proven Protease Inhibitor. Cell, 2020. 181(2): p. 271-280 e8.
7. Kumar, A. A.; et al. Convenient Syntheses of 1,2-trans Selenoglycosides Using Isoselenuronium Salts as Glycosylselenenyl Transfer Reagents. Carbohydrate Research. 2012, 360, 8-18.
8. Laine, R. A., A calculation of all possible oligosaccharide isomers both branched and linear yields 1.05×10(12) structures for a reducing hexasaccharide: the Isomer Barrier to development of single-method saccharide sequencing or synthesis systems. Glycobiology, 1994. 4(6): p. 759-67.
9. Liu, Y.; et al. Sodium Selenosulfate from Sodium Sulfite and Selenium Powder: An Odorless Selenylating Reagent for Alkyl Halides to Produce Dialkyl Diselenide Catalysts. Synthetic Letters. 2019, 30 (14), 1698-1702.
10. Lu, L.; et al. Catalyst and Additive-Free Selective Ring-Opening Selenocyanation of Heterocycles with Elemental Selenium and TMSCN. Advanced Synthesis & Catalysis. 2021, 363 (5), 1346-1351.
11. Moore, M. J., et al., Retroviruses pseudotyped with the severe acute respiratory syndrome coronavirus spike protein efficiently infect cells expressing angiotensin-converting enzyme 2. J Virol, 2004. 78(19): p. 10628-35.
12. Musarrat, F., et al., The Amino Terminus of Herpes Simplex Virus 1 Glycoprotein K (gK) Is Required for gB Binding to Akt, Release of Intracellular Calcium, and Fusion of the Viral Envelope with Plasma Membranes. J Virol, 2018. 92(6).
13. Musarrat, F., et al., The anti-HIV drug nelfinavir mesylate (Viracept) is a potent inhibitor of cell fusion caused by the SARSCoV-2 spike (S) glycoprotein warranting further evaluation as an antiviral against COVID-19 infections. J Med Virol, 2020. 92(10): p. 2087-2095.
14. Nasim, M.; et al. Aromatic Selenocyanates as a Unique Class of Non-Mutagenic Antimicrobial Selenium Compounds with Pronounced Activity Against Multidrug Resistant ESKAPE Bacteria. New Journal of Chemistry. 2019, 43, 6021-6031.
15. Plano, D.; et al. Selenocyanates and Diselenides: A New Class of Potent Antileishmanial Agents. European Journal of Medicinal Chemistry. 2011, 46 (8), 3315-3323.
16. Shajahan, A., et al., Comprehensive characterization of N- and O-glycosylation of SARS-CoV-2 human receptor angiotensin converting enzyme 2. Glycobiology, 2021. 31(4): p. 410-424.
17. Shajahan, A., et al., Deducing the N- and O-glycosylation profile of the spike protein of novel coronavirus SARS-CoV-2. Glycobiology, 2020. 30(12): p. 981-988.
18. Wang, S. S., et al., Thioglycosides Are Efficient Metabolic Decoys of Glycosylation that Reduce Selectin Dependent Leukocyte Adhesion. Cell Chem Biol, 2018. 25(12): p. 1519-1532 e5.
19. Xia, S., et al., Fusion mechanism of 2019-nCoV and fusion inhibitors targeting HR1 domain in spike protein. Cell Mol Immunol, 2020. 17(7): p. 765-767.
20. Xia, S., et al., Inhibition of SARS-CoV-2 (previously 2019-nCoV) infection by a highly potent pan-coronavirus fusion inhibitor targeting its spike protein that harbors a high capacity to mediate membrane fusion. Cell Res, 2020. 30(4): p. 343-355.
21. Yan, R., et al., Structural basis for the recognition of SARS-CoV-2 by full-length human ACE2. Science, 2020. 367(6485): p. 1444-1448.
22. Yang, Q., et al., Inhibition of SARS-CoV-2 viral entry upon blocking N- and O-glycan elaboration. Elife, 2020. 9.
23. Zhao, X., et al., Inhibition of endoplasmic reticulum-resident glucosidases impairs severe acute respiratory syndrome coronavirus and human coronavirus NL63 spike protein-mediated entry by altering the glycan processing of angiotensin I-converting enzyme 2. Antimicrob Agents Chemother, 2015. 59(1): p. 206-16.

1. A glycoside comprising Formula I or Formula II: 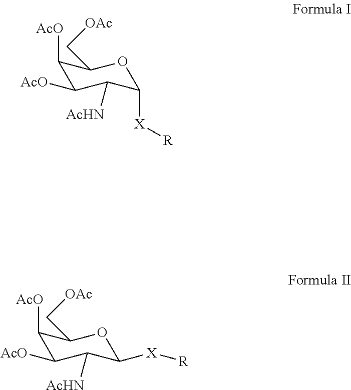
wherein X is selected from O, S, or Se;
and wherein R is selected from C1-C20 linear or branched alkyl, alkenyl, aryl, alkylaryl, or arylalkyl.

2. The glycoside of claim 1, wherein the glycoside has Formula I.

3. The glycoside of claim 1, wherein the glycoside has Formula II.

4. The glycoside of claim 1, wherein X is O.

5. The glycoside of claim 1, wherein X is S.

6. The glycoside of claim 1, wherein X is Se.

7. The glycoside of claim 1, wherein R is selected from geranyl, benzyl, methyl naphthyl, octyl, naphthyl, or any combination thereof.

8. The glycoside of claim 1, wherein the glycoside is selected from N,O-tetra-acetyl-GlcNAc-β-S-geranyl, N,O-tetra-acetyl-GlcNAc-β-S-benzyl, N,O-tetra-acetyl-GlcNAc-β-S-octyl, N,O-tetra-acetyl-GlcNAc-β-S-naphthyl, N,O-tetra-acetyl-GlcNAc-β-S-methylnaphthyl, N,O-tetra-acetyl-GalNAc-α-O-benzyl, N,O-tetra-acetyl-GalNAc-α-O-methylnaphthyl, N,O-tetra-acetyl-GalNAc-α-S-benzyl, N,O-tetra-acetyl-GalNAc-α-S-methylnaphthyl, N,O-tetra-acetyl-GalNAc-α-S-geranyl, N,O-tetra-acetyl-GalNAc-α-S-octyl, N,O-tetra-acetyl-GalNAc-α-S-naphthyl, N,O-tetra-acetyl-GlcNAc-β-Se-geranyl, N,O-tetra-acetyl-GlcNAc-β-Se-methylnaphthyl, N,O-tetra-acetyl-GalNAc-α-Se-benzyl, N,O-tetra-acetyl-GalNAc-α-Se-methylnaphthyl, N,O-tetra-acetyl-GlcNAc-β-Se-naphthyl, N,O-tetra-acetyl-GlcNAc-β-Se-octyl, N,O-tetra-acetyl-GalNAc-α-Se-octyl, or any combination thereof.

9. A pharmaceutical composition comprising the compound of claim 1.

10. A method for treating or preventing at least one symptom of a virus in a subject, the method comprising administering the compound of claim 1 to the subject.

11. The method of claim 10, wherein the subject is a mammal.

12. The method of claim 11, wherein the mammal is a human, dog, cat, goat, sheep, horse, cattle, swine, hamster, rat, mouse, guinea pig, or rabbit.

13. The method of claim 10, wherein the virus is a herpes virus, respiratory syncytial virus (RSV), an influenza virus, or a coronavirus.

14. The method of claim 13, wherein the herpes virus is HSV-1 or HSV-2.

15. The method of claim 13, wherein the coronavirus is SARS-CoV-2.

16. The method of claim 10, wherein the compound or pharmaceutical composition is administered topically to at least one infected site.

17. The method of claim 10, wherein the compound or pharmaceutical composition is formulated as a cream, gel, salve, or eye drop.

18. The method of claim 10, wherein the method does not cause systemic side effects in the subject.

19. The method of claim 10, wherein the glycoside interferes with host cell glycan decoration of viral proteins.

20. The method of claim 10, wherein the method does not cause the virus to mutate.
